(19) United States
(12) Patent Application Publication
(10) Pub. No.: US 2015/0290384 A1
(43) Pub. Date: Oct. 15, 2015

(54) ADSORBENT FOR DIALYSIS

(71) Applicant: TRIOMEM AB, Lund (SE)

(72) Inventor: Carin Malmborg, Hoor (SE)

(73) Assignee: TRIOMED AB, Lund (SE)

(21) Appl. No.: 14/646,837

(22) PCT Filed: Nov. 22, 2013
(86) PCT No.: PCT/SE2013/000183

§ 371 Date: May 22, 2015

(30) Foreign Application Priority Data
Nov. 23, 2012 (SE) 1230131-3

(51) Int. Cl. A61M 1/36; A61M 1/16

(52) U.S. Cl. CPC A61M 1/3679; A61M 1/16

(57) ABSTRACT

A regeneration circuit for regeneration of a dialysis fluid. An inlet and an outlet are arranged to pass a dialysis fluid to and from the regeneration circuit by means of a pump. An adsorbent cartridge is arranged downstream of the pump and comprises an adsorbent made of e.g. activated carbon and an adsorbent made of metal-complexed chitosan and possibly further adsorbents. Downstream of the last adsorbent is arranged a further general adsorbent arranged to adsorb metal ions, such as copper ions, possibly given off by previous adsorbents. Such a general adsorbent is an adsorbent made of uncomplexed chitosan, which adsorb metal ions and does not adsorb physiological electrolytes commonly included in a dialysis fluid, such as sodium, potassium, calcium and magnesium ions.

### FIELD OF INVENTION

The present invention relates to an adsorbent for dialysis fluids for use in treatment of renal diseases.

### BACKGROUND

Patients suffering from renal diseases need dialysis treatment. There are two different modalities of dialysis, namely hemodialysis and peritoneal dialysis. In hemodialysis, blood from the patient is circulated in an extracorporeal circuit into contact with one side of a semi-permeable membrane of a dialyzer, the other side being in contact with a dialysis fluid. Substances are transferred over the membrane via diffusion and convection. In peritoneal dialysis, the dialyzer membrane is in principle replaced by an endogenous membrane, namely the peritoneal membrane of the patient.

During dialysis, large quantities of dialysate are consumed. The spent dialysate is normally discarded. In order to reduce the amount of used liquid, the spent dialysis fluid may be regenerated by adsorption of certain substances by an adsorption cartridge. Most previously known adsorption cartridges use activated carbon for removal of many unwanted substances. However, activated carbon may be inefficient in adsorbing urea. In addition, activated carbon cannot adsorb certain electrolytes, such as sodium, potassium, magnesium or calcium ions.

In order to adsorb urea, one previously used method is to pass the spent dialysate through a column comprising urease, which converts urea into ammonium and carbonate ions. The ammonium is removed by for example zirconium phosphate. However, residual ammonium may be toxic to the patient and may increase the pH. Other methods of removing urea are highly desired.

Another promising adsorbent for removing urea from body-fluids is copper(II)-chitosan, as suggested in an article: “Preparation and Characterization of Chitosan/Cu(II) Affinity Membrane for Urea Adsorption”, by Jiahao Liu, Xin Chen, Zhengzhong Shao, Ping Zhou, published in Journal of Applied Polymer Science, Vol. 90, 1108-1112 (2003).

If an adsorbent made of copper(II)-chitosan is used, there is a risk that copper-ions are released from the adsorbent and passes further to the dialysis membrane or the peritoneal cavity. Copper is an essential metal required by the body in small quantities. However, when people are exposed to (oral) copper levels of above 1.3 mg/l for short periods of time, stomach and intestinal problems may occur. Long-term exposure to high level of copper ions may lead to kidney and liver damage, as well as accumulation in the brain (Wilson's disease).

Other metal ions may be present in the dialysis fluid, and such metal ions may be desired to be removed.

Thus, there is a need for an adsorbent for removal of copper ions and other metal ions, especially when an adsorbent made of copper(II)-chitosan is used for urea removal. At the same time, the important electrolytes of a dialysis fluid should not be influenced upon. Such electrolytes are sodium, potassium, calcium and magnesium ions and corresponding negatively charged ions, such as chloride ions. In addition, further components, such as bicarbonate and/or acetate should not be adsorbed as well as glucose or icodextrin or any other osmotically or oncotically active agent used in peritoneal dialysis. In addition, the pH should not be compromised.

### SUMMARY OF THE INVENTION

Accordingly, an object of the present invention is to mitigate, alleviate or eliminate one or more of the above-identified deficiencies and disadvantages singly or in any combination.

In an aspect, there is provided an adsorbent cartridge for regeneration of a dialysis fluid, wherein the adsorption cartridge comprises an adsorbent made of metal-complexed chitosan for adsorption of substances from the dialysis fluid circulated through the adsorbent cartridge, characterized in that an adsorbent made of uncomplexed chitosan is arranged downstream of the adsorbent made of metal-complexed chitosan for adsorption of metal ions released from the adsorbent made of metal-complexed chitosan.

In an embodiment, the adsorbent cartridge may comprise further adsorbents, such as activated carbon, arranged upstream of the adsorbent made of uncomplexed chitosan.

In a further embodiment, the adsorbent made of metal-complexed chitosan and the adsorbent made of uncomplexed chitosan may be arranged in one and the same adsorbent cartridge in said order. The adsorbent made of metal-complexed chitosan may be made of at least one of Cu(II)-chitosan, Fe(III)-chitosan, Zn(II)-chitosan, La(III)-chitosan, Cr(III)-chitosan, or combinations thereof.

In another aspect, there is provided a regeneration circuit for regeneration of a dialysis fluid, comprising an inlet and an outlet arranged to pass a dialysis fluid to and from the regeneration circuit; a pump arranged to pump the fluid through the circuit from the inlet to the outlet; and an adsorbent cartridge comprising an adsorbent made of metal-complexed chitosan; characterized by an adsorbent made of uncomplexed chitosan arranged downstream of the adsorbent made of metal-complexed chitosan.

In an embodiment, the adsorbent made of metal-complexed chitosan and the adsorbent made of uncomplexed chitosan are arranged in one and the same cartridge.

In a further embodiment, the regeneration circuit may further comprise a replacement solution cartridge arranged downstream of the adsorbent cartridge for addition of replacement solutions to the dialysis fluid, wherein the adsorbent made of uncomplexed chitosan is arranged at the replacement solution cartridge. The replacement solutions may be arranged to be delivered to the dialysis fluid, either before, after or into the adsorbent made of uncomplexed chitosan. In addition, a sterile filter may be arranged upstream of the outlet, wherein the adsorbent made of uncomplexed chitosan may be arranged at or adjacent the sterile filter upstream of the sterile filter.

### BRIEF DESCRIPTION OF THE DRAWINGS

Further objects, features and advantages of the invention will become apparent from the following detailed description of embodiments of the invention with reference to the drawings, in which:

FIG. 1 is a schematic diagram of a first embodiment of a dialysis regeneration system according to the present invention.

FIG. 2 is a schematic diagram similar to FIG. 1 of a second embodiment of the dialysis regeneration system.

FIG. 3 is a schematic diagram similar to FIG. 1 of a third embodiment of the dialysis regeneration system.

FIG. 4 is a structure scheme of chitosan molecules in complex with a copper(II) ion, which can bind up to two molecules of urea.

### DETAILED DESCRIPTION OF EMBODIMENTS

Below, several embodiments of the invention will be described. These embodiments are described in illustrating purpose in order to enable a skilled person to carry out the invention and to disclose the best mode. However, such embodiments do not limit the scope of the invention. Moreover, certain combinations of features are shown and discussed. However, other combinations of the different features are possible within the scope of the invention.

In hemodialysis, the spent dialysate is normally discarded, resulting in consumption of large volumes of water (up to hundred liters per treatment of 4 hours) of high purity. Often, reverse osmosis water is used, which is expensive to produce in large quantities. In addition, a reverse osmosis apparatus is cumbersome and takes up a large space and produces noise during use thereof.

In peritoneal dialysis, the peritoneal dialysis fluid is normally sterilized, for example by autoclaves. This procedure also adds to the costs and complexity.

In order to reduce the amount of dialysis fluid required, the dialysis fluid may be regenerated by passing the dialysis fluid through an adsorption cartridge and reuse of the regenerated fluid. The adsorbent cartridge most often comprises activated carbon, which is effective for removal of many undesired waste products or metabolite products from a dialysis fluid, including uric acid. However, activated carbon is ineffective in removal of urea, which is a metabolic product that should be removed in amounts of up to about 15 g per day.

In a hemodialysis system, the regeneration circuit may be arranged as shown in the document GB1484642A, which discloses a system comprising urease for catalytic conversion of urea to ammonium and carbonate ions. The ammonium ions are adsorbed by a zeolite, such as phillipsite loaded with sodium ions. Calcium ions are added after the phillipsite cartridge to precipitate calcium carbonate.

Another promising substance for removing urea from body-fluids is copper(II)-chitosan, as suggested in the article mentioned above.

In the manufacturing process, chitosan is contacted with copper ions, wherein copper ions are complexed to the chitosan polymer amine groups as shown in FIG. 4. Each copper ion can complex with one, two, three or four amine groups, preferably two amine groups of the chitosan polymer, giving cross-linking, and stabilization of the porous chitosan membrane is achieved.

However, a copper(II)-chitosan material may release copper ions when arranged in a liquid environment. Copper ions in the dialysis fluid should be avoided, and thus, such released copper ions need to be removed.

In addition, iron(III)-chitosan may be used for urea adsorption, during which iron ions may be released. Iron ions in the dialysis fluid should be avoided, and thus, such released iron ions need to be removed. Other metal-complexed chitosans that may be used are at least one of Zn(II)-chitosan, Cd(II)-chitosan, Co(II)-chitosan, Al(III)-chitosan, La(III)-chitosan, Cr(III)-chitosan or a combination of any one of them.

However, an adsorbent for the mentioned metal ions should not influence upon other ions, which should be present in the dialysis fluid, such as sodium, potassium, calcium and magnesium ions as well as bicarbonate, acetate, lactate, chloride ions and an osmotically or oncotically active agent such as glucose or icodextrin. In addition, desired proteins, such as albumin should not be adsorbed.

Uncomplexed chitosan may be used as an adsorbent. Uncomplexed chitosan is a chitosan where the NH₂ group is not linked to a metal ion, contrary to a metal-complexed chitosan, which is complexed with a metal ion, such as copper(II).

The first transition metal ions are collected by uncomplexed chitosan with the notable exception of manganese, because they form oxyanions (titanate, vanadate and chromate): vanadate is collected on uncomplexed chitosan at the impressive ratio of 2.3:1 by weight in the product obtained. In addition, previously reported studies on uncomplexed chitosan show that the binding followed the pattern: Cu²⁺>Fe²⁺>Zn²⁺>Cd²⁺. No detectable binding of Mg²⁺ and Ca²⁺ with chitosan was found. In another study of metal ion binding to uncomplexed chitosan, a similar order of binding was reported: Cu²⁺>Zn²⁺>Cd²⁺>Co²⁺>Fe³⁺. In addition, Fe²⁺, Cd²⁺, Al³⁺, Ni²⁺, Ag²⁺ are bound to uncomplexed chitosan.

Moreover, the uncomplexed chitosan is able to bind other substances which are unwanted in a medical environment, should they accidentally enter into the system, for examples in plastic materials used in the system or connectors or fluids used. For example mercury and lead (and uranium) are such substances that are bound to uncomplexed chitosan.

In addition, we have found that uncomplexed chitosan has no detectable binding of potassium ions and only a small binding of sodium ions. The same is true for the negatively charged ions of a dialysis fluid. However, uncomplexed chitosan may bind a small amount of glucose, but no detectable amounts of creatinine, phosphate or beta2-micro-globulin, which is a small protein.

By using uncomplexed chitosan as adsorbent for metal ions, such as copper or iron ions, the dialysis fluid may be depleted of such metal ions.

Uncomplexed chitosan is a chitosan where the NH₂ group is not linked to a metal ion, contrary to the copper(II)-chitosan shown in FIG. 4, which is complexed with copper(II).

Uncomplexed chitosan which may be used in the present embodiments are such chitosans that have a deacetylation degree of more than 50%, such as more than 80%, for example above 90%.

In order to examine the suitability of uncomplexed chitosan to adsorb copper ions in a dialysis solution, the following experiment was conducted.

### EXAMPLE

20.0 g ground wet copper(II)-chitosan was put in 100 ml peritoneal dialysis solution with following composition:

| | | |
|---|---|---|
| Glucose | 75.5 | mmole/L |
| Sodium | 132 | mmole/L |
| Calcium | 1.25 | mmole/L |
| Magnesium | 0.25 | mmole/L |
| Chloride | 93.5 | mmole/L |
| Bicarbonate | 25 | mmole/L |
| Lactate | 15 | mmole/L |
| Urea | 21.7 | mmole/L |

Sample 1 was taken as reference sample of the original solution and sample 2 was taken after 6 hours, after which 11.0 gram wet uncomplexed chitosan without copper was added to adsorb free copper ions, and sample 3 was taken after 18 hours.

| | urea | copper |
|---|---|---|
| Sample | mmole/L | mg/L |
| 1 | 19.8 | 0 |
| 2 | 16.7 | 4.6 |
| 3 | 15.6 | 0.5 |

From the above experiment, it can be concluded that copper(II)-chitosan gives off copper ions to the peritoneal dialysis fluid (Sample 2). In addition, it is shown that uncomplexed chitosan is able to remove the copper ions to a concentration below 0.6 mg/L (Sample 3). Moreover, the uncomplexed chitosan may be effective to remove some additional urea, which has escaped adsorption by copper(II)-chitosan. The amount of uncomplexed chitosan used as copper adsorbent is from 1 to 60% of the copper(II)-chitosan.

### Example 2

3.2 g of uncomplexed chitosan felt was put in a Plexiglas cylinder. A solution containing 2.2 mg/L Cu (5 mg/L CuSO₄) in deionized water was pumped through the cylinder at 16 ml/minute, during 5 hours. The initial measuring point is higher than the following because it takes some time to wet the chitosan felt. During 2 hours the Cu concentration after the chitosan felt filter was not exceeding 0.1 mg/L.

| Time (hours) | Cu mg/L |
|---|---|
| 0 | 2.1133 |
| 0.01 | 0.0800 |
| 0.25 | 0.0033 |
| 0.5 | 0.0067 |
| 1 | 0.0200 |
| 1.5 | 0.0533 |
| 2 | 0.0967 |
| 2.5 | 0.1317 |
| 3 | 0.1767 |
| 3.5 | 0.2200 |
| 4 | 0.2683 |
| 5 | 0.3583 |

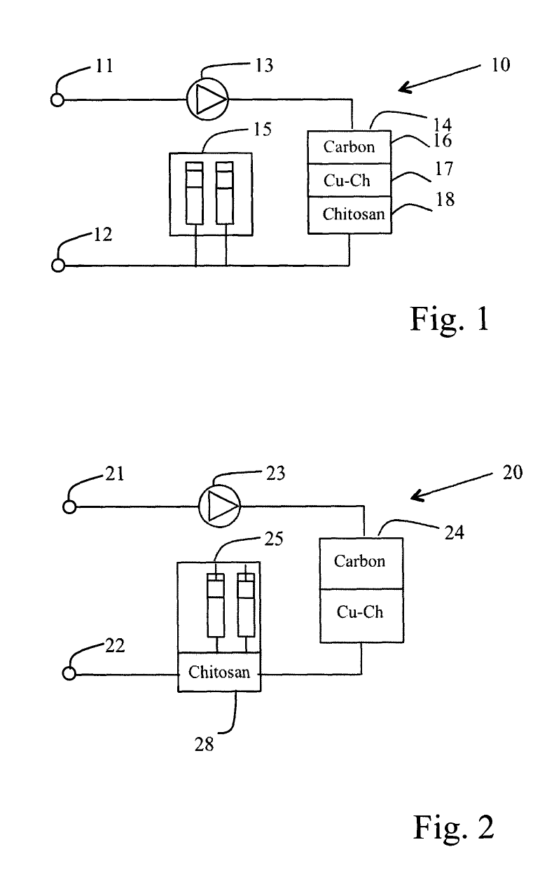

FIG. 1 is a schematic diagram of a first embodiment of a regeneration device. The regeneration device 10 comprises an inlet 11 for a dialysis fluid and an outlet 12 for regenerated dialysis fluid. The inlet and outlet may be connected to a dual lumen peritoneal dialysis catheter installed in a patient. The peritoneal cavity of the patient comprises peritoneal dialysis fluid, which should be regenerated with the adsorption device of FIG. 1.

Alternatively, the inlet 11 and outlet 12 may be connected to a dialyzer for hemodialysis, hemofiltration or hemodiafiltration.

From the inlet 11, the fluid passes via a line to a pump 13, which may be a peristaltic pump. From the pump, the fluid passes to an adsorption cartridge 14. From the cartridge 14, the fluid passes via a line to the outlet 12. A cartridge 15 comprising one or several replacement solutions may be arranged to add replacement solutions to the outgoing dialysis fluid. So far, the regeneration device 10 is similar to previously known technique.

As is also conventional, the cartridge 14 may comprise several adsorbents. One adsorbent, which is included in almost every regeneration systems, is a first adsorbent 16 comprising activated carbon.

In addition, there is a urea adsorbent 17 comprising copper(II)-chitosan, which is effective in adsorption of urea and also some other substances, such as phosphate and creatinine.

Furthermore, there is arranged a general adsorbent 18 for metal ions, such as copper or iron ions. The general adsorbent may comprise uncomplexed chitosan.

There may be further adsorbents included in the adsorbent cartridge.

The general adsorbent 18 for adsorption of metal ions, should be arranged downstream of the urea adsorbent 17, since it is contemplated that the general adsorbent should inter alia adsorb copper ions released from the adsorbent 17 comprising copper(II)-complexed chitosan. The general adsorbent 18 may also be effective for adsorption of other substances or ions in the dialysis fluid, such as urea and phosphate that has escaped the urea adsorbent 17, and other metal ions, which may have been released by any component before the general adsorbent, such as the first adsorbent 16, or may have been removed from the patient blood during the dialysis treatment, such as iron ions. The general adsorbent 18 should not adsorb any physiological electrolytes, such as Na⁺, Ca²⁺, Mg²⁺ and Cl⁻

Different orders of the adsorbents 16, 17, 18 may be used. Thus, the urea adsorbent 17 may be arranged before the first adsorbent 16 comprising activated carbon.

However, in one embodiment, the general adsorbent 18 may be arranged downstream of all the other adsorbents.

In another embodiment, the general adsorbent 18 is arranged after the urea adsorbent 17 and the first absorbent 16 is arranged after the general adsorbent 18. Further arrangements with still further adsorbents may be used.

The replacement solution cartridge 15 may be arranged to add replacement solutions such as glucose and electrolytes.

FIG. 2 discloses another embodiment of the regeneration device 20. In this second embodiment, the general adsorbent is arranged in the same cartridge as the replacement solutions. By this arrangement, it is assured that the general adsorbent is arranged downstream of all the other adsorbents.

The second embodiment of the regeneration device comprises an inlet 21, an outlet 22, a pump 23, an adsorbent cartridge 24 and a replacement solution cartridge 25 similar to the first embodiment. The general adsorbent 28 is arranged in the replacement solution cartridge 25.

The replacement solutions may be arranged to be introduced before the general adsorbent, inside the general adsorbent or after the general adsorbent. If the replacement solution is introduced before the general adsorbent, any contamitants in the replacement solutions may be adsorbed by the general adsorbent. If the replacement solution is introduced after the general adsorbent, the fluid will be independent of the general adsorbent.

One replacement solutions, such as the electrolyte replacement solution, may be introduced before or inside the general adsorbent, while another replacement solution, such as the glucose replacement solution may be arranged to be added after the general adsorbent. The latter arrangement is advantageous if the general adsorbent adsorbs some glucose.

The arrangement of the general adsorbent 28 at the replacement solution cartridge 25 makes it possible to replace the adsorbent cartridge 24 without replacing the general adsorbent 28. It is also possible to replace the general adsorbent 28 together with the replacement solution cartridge 25 independently of the adsorption cartridge 24.

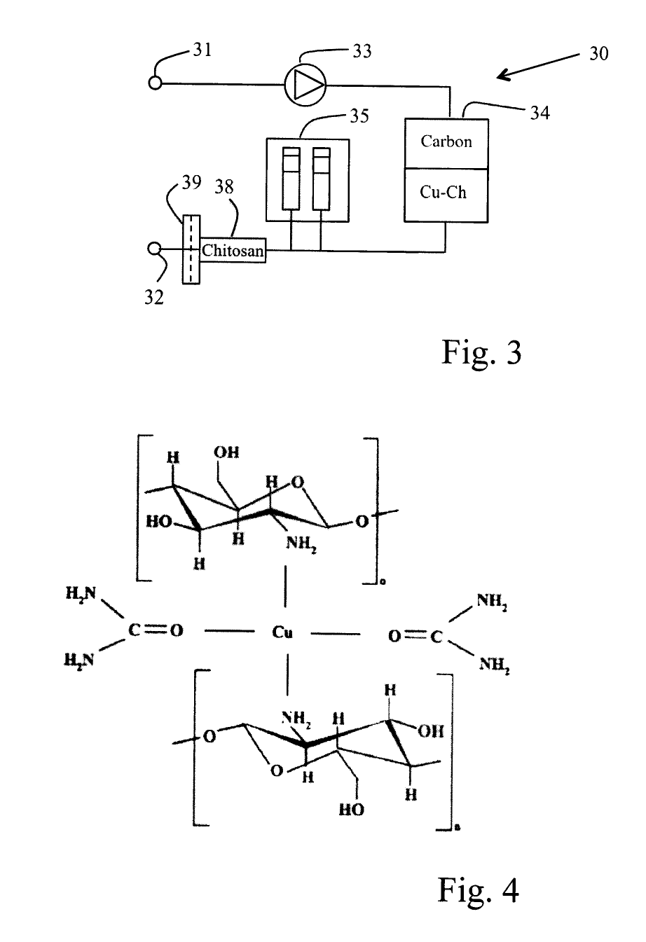

FIG. 3 discloses a further embodiment of the regeneration device 30. In this device, the general adsorbent is arranged in connection with a filter or sterile filter arranged immediately upstream of the outlet 32 to the dialyzator or the peritoneal dialysis catheter.

The regeneration device 30 of FIG. 3 comprises an inlet 31, an outlet 32, a pump 33, an adsorbent cartridge 34 and a replacement solution cartridge 35 similar to the first embodiment of FIG. 1. A sterile filter 39 is arranged upstream of the outlet 32. The general adsorbent 38 is arranged in connection with the sterile filter 39, just upstream of the sterile filter or in combination with the sterile filter. In this arrangement, all dialysis fluids including replacement solutions pass through the general adsorbent 38.

In the claims, the term “comprises/comprising” does not exclude the presence of other elements or steps. Furthermore, although individually listed, a plurality of means, elements or method steps may be implemented by e.g. a single unit. Additionally, although individual features may be included in different claims or embodiments, these may possibly advantageously be combined, and the inclusion in different claims does not imply that a combination of features is not feasible and/or advantageous. In addition, singular references do not exclude a plurality. The terms “a”, “an”, “first”, “second” etc. do not preclude a plurality. Reference signs in the claims are provided merely as a clarifying example and shall not be construed as limiting the scope of the claims in any way.

Although the present invention has been described above with reference to specific embodiment and experiments, it is not intended to be limited to the specific form set forth herein. Rather, the invention is limited only by the accompanying claims and, other embodiments than those specified above are equally possible within the scope of these appended claims.

1. An adsorbent cartridge for regeneration of a dialysis fluid, wherein
the adsorption cartridge comprises an adsorbent made of metal-complexed chitosan for adsorption of substances from the dialysis fluid circulated through the adsorbent cartridge,
wherein
an adsorbent made of uncomplexed chitosan is arranged downstream of the adsorbent made of metal-complexed chitosan for adsorption of metal ions in the dialysis fluid released from the adsorbent made of metal-complexed chitosan.

2. The adsorbent cartridge according to claim 1, wherein the adsorbent cartridge comprises further adsorbents, such as activated carbon, arranged upstream of the adsorbent made of uncomplexed chitosan.

3. The adsorbent cartridge according to claim 1, wherein the adsorbent made of metal-complexed chitosan and the adsorbent made of uncomplexed chitosan are arranged in one and the same adsorbent cartridge in said order.

4. The adsorbent cartridge according to claim 1, wherein the adsorbent made of metal-complexed chitosan is made of at least one of Cu(II)-chitosan, Fe(III)-chitosan, Zn(II)-chitosan, La(III)-chitosan, Cr(III)-chitosan, or combinations thereof.

5. A regeneration circuit for regeneration of a dialysis fluid, comprising
an inlet and an outlet arranged to pass a dialysis fluid to and from the regeneration circuit;
a pump arranged to pump the fluid through the circuit from the inlet to the outlet; and
an adsorbent cartridge comprising an adsorbent made of metal-complexed chitosan;
further comprising an adsorbent made of uncomplexed chitosan arranged downstream of the adsorbent made of metal-complexed chitosan.

6. The regeneration circuit according to claim 5, in which the adsorbent made of metal-complexed chitosan and the adsorbent made of uncomplexed chitosan are arranged in one and the same cartridge.

7. The regeneration circuit according to claim 5, further comprising
a replacement solution cartridge arranged downstream of the adsorbent cartridge for addition of replacement solutions to the dialysis fluid, wherein the adsorbent made of uncomplexed chitosan is arranged at the replacement solution cartridge.

8. The regeneration circuit according to claim 7, wherein the replacement solutions are arranged to be delivered to the dialysis fluid, either before, after or into the adsorbent made of uncomplexed chitosan.

9. The regeneration circuit according to claim 5, further comprising a sterile filter arranged upstream of the outlet, wherein the adsorbent made of uncomplexed chitosan is arranged at or adjacent the sterile filter upstream of the sterile filter.
